(19) United States
(12) Patent Application Publication
(10) Pub. No.: US 2014/0171711 A1
(43) Pub. Date: Jun. 19, 2014

(54) METHOD OF MAKING PARA-DIETHYLBENZENE

(71) Applicant: KING FAHD UNIVERSITY OF PETROLEUM AND, Dhahran (SA)

(72) Inventor: SULAIMAN SALEH AL-KHATTAF, DHAHRAN (SA)

(73) Assignee: KING FAHD UNIVERSITY OF PETROLEUM AND MINERALS, DHAHRAN (SA)

(21) Appl. No.: 13/714,278

(22) Filed: Dec. 13, 2012

(51) Int. Cl. C07C 2/66

(52) U.S. Cl. CPC C07C 2/66
USPC 585/467

(57) ABSTRACT

The method of making para-diethylbenzene selectively produces para-diethylbenzene by reacting ethylbenzene and ethanol over a zeolite catalyst, such as ZSM-5. The zeolite catalyst is first silylated, preferably through multiple silylation. The silylated zeolite catalyst is then partially coked to form a treated catalyst. The treated catalyst is placed in argon gas in a reaction chamber, and the argon gas is flowed over the treated catalyst. A feedstock mixture of ethylbenzene and ethanol in a molar ratio of 1:1 is then injected into the reaction chamber at a temperature of about 300° C. to produce the para-diethylbenzene, which is then removed from the reaction chamber.

### BACKGROUND OF THE INVENTION

1. Field of the Invention

The present invention relates generally to the manufacture of para-diethylbenzene, and particularly to a method of making para-diethylbenzene by reacting ethylbenzene and ethanol over a zeolite catalyst that has been silylated and pre-treated by coking.

2. Description of the Related Art

Aromatic conversion reactions are of great industrial interest and importance. Such reactions include alkylation and transalkylation used to produce alkylaromatics, such as ethylbenzene, diethylbenzene, para-diethylbenzene, ethyltoluene, cumene and higher aromatics. Dialkylbenzenes, such as xylene, diethylbenzene and para-diethylbenzene are of particular importance, as they are used for the production of polyesters, solvents, photodevelopers and antioxidants. Diethylbenzenes are also used as solvents and precursors for cross-linking agents in producing resins.

A wide variety of reactor systems have been developed for carrying out aromatic conversion reactions. Conventional aromatic conversion systems include fixed bed reactors (such as multi-tubular fixed bed reactors) and fluidized bed reactors. The alkylation of ethylbenzene with ethanol is a reaction that is of immense industrial importance. This reaction provides an alternate route for producing various isomers of diethylbenzene (ortho-, meta- and para-diethylbenzene). Due to the rapid development of biochemical engineering technology, the cost of obtaining ethanol has greatly decreased. Thus, the direct use of ethanol in manufacturing diethylbenzene is of economic benefit to those countries where biomass-derived alcohol is readily available for manufacturing chemicals. Additionally, in situ dehydration of alcohols leads to prolonged catalyst activity, since the water of reaction suppresses coke formation, which is in contrast to vapor phase alkylation with ethylene at higher temperatures, where significant coke formation typically occurs.

Alkylation of ethylbenzene to diethylbenzene and para-diethylbenzene is commonly performed as an acid-catalyzed process. Diethylbenzene is conventionally synthesized by using existing alkylation catalysts, such as AlCl₃, HF, BF₃, and the like. Due to the strong acidity of these catalysts, disposal of the selected catalyst causes serious environmental pollution, along with corrosion of equipment used during the manufacturing process.

Thus, a method of making para-diethylbenzene solving the aforementioned problems is desired.

### SUMMARY OF THE INVENTION

The method of making para-diethylbenzene involves reacting ethylbenzene and ethanol over a zeolite catalyst, such as ZSM-5. The zeolite catalyst is first silylated, preferably through multiple silylation. The silylated zeolite catalyst is then partially coked to form a treated catalyst. The treated catalyst is placed in argon gas in a reaction chamber, and the argon gas is flowed over the treated catalyst. A feedstock mixture of ethylbenzene and ethanol in a molar ratio of 1:1 is then injected into the reaction chamber at a temperature of about 300° C. to produce the para-diethylbenzene, which is then removed from the reaction chamber.

These and other features of the present invention will become readily apparent upon further review of the following specification.

### DETAILED DESCRIPTION OF THE PREFERRED EMBODIMENTS

The method of making para-diethylbenzene (p-DEB) relates generally to the manufacture of diethylbenzene, and particularly to a method that is selective for para-diethylbenzene by reacting ethylbenzene and ethanol over a zeolite catalyst. The zeolite catalyst is first heated in argon gas within a reaction chamber. The zeolite catalyst may be one of ZSM-5, ZSM-11, ZSM-12, ZSM-22 or ZSM-23, and preferably has a silica-to-alumina molar ratio (SiO₂:Al₂O₃) between about 50 and about 150. In the preferred embodiment, ZSM-5 is used as the zeolite catalyst (with a SiO₂:Al₂O₃ molar ratio of 80). The catalyst is then modified by silylation. Between four and ten silylations may be used for the modification, although experimentally the optimal number of silylations has been found to be six. As is known in the art, silylation may be carried out in the vapor phase or the liquid phase. The zeolite is impregnated with an organo silicon compound that is dissolved or dispersed in a carrier or solvent, followed by calcination of the treated zeolite in an oxygen-containing atmosphere under conditions sufficient to remove organic material therefrom and deposit siliceous material. Such silylation results in deposition of at least 1% by weight of siliceous material on the catalyst. In the present “multiple silylation” process, the zeolite is calcined after each impregnation of organosilicon compound. Preferably, the catalyst is finely powdered in order to facilitate continuous and easy fluidization.

Any suitable type of reactor having a reaction chamber, a gas inlet and a gas outlet may be utilized, such as a riser simulator or fast fluidized-bed reactor. Fast fluidized-bed reactors typically include a metallic gasket that seals a pair of chambers, and an impeller located within an upper chamber. Upon rotation of a shaft, gas is forced outward from the center of the impeller towards the walls of the chamber. This creates a lower pressure in the center region of the impeller, thus inducing a flow of gas upward through a catalyst chamber from the bottom of the reactor, where the pressure is slightly higher. The impeller provides a fluidized-bed of catalyst particles, as well as intense gas mixing inside the reactor. Preferably, such a reactor is used in the present method of making para-diethylbenzene. Generally, the reactor should allow homogenous mixing of solid catalyst in the fluidized form and the reactant in vapor form for a short contact reaction time. The reaction time may be between ten and thirty seconds, with an optimal reaction time of about twenty seconds.

The reactor is preferably equipped with a heating facility in order to attain near isothermal conditions inside the reactor at reaction temperatures in the range of about 250° C. to about 450° C. Experimentally, the optimal temperature has been found to be about 300° C. Prior to the reaction, the modified catalyst is pre-treated by partial coking in order to fully utilize the potential of the catalyst to obtain very high selectivity of para-diethylbenzene. The partial coking is carried out under controlled conditions, so that the level of coke on the catalyst is in the range of about 0.7 wt % to about 1.5 wt %. The preferred level of coke is about 1.0 wt %.

Following the pre-treatment of the modified zeolite catalyst, the modified zeolite catalyst is reacted in the fluidized-bed reactor, using a feedstock stream containing ethylbenzene. The feedstock stream may also include an alkylating agent, such as ethanol. The reaction temperatures is in the range of about 250° C. to about 450° C., the optimal temperature being found to be about 300° C. The pressure is in the range of about 0.1 atm to about 5.0 atm, the optimal pressure being found to be about 1.0 atm.

The product stream is then separated by distillation, as is known in the art, and the unconverted feedstock is preferably recycled. This may be performed in a product separator unit using conventional distillation to separate the product species according to their boiling points. The spent catalyst is continuously partially regenerated under controlled conditions in order to maintain the activity and selectivity of the catalyst. Conditions should be carefully controlled to achieve the necessary level of coke on the regenerated catalyst. The level of coke on the catalyst is preferably in the range of 0.7 wt % to 1.5 wt %, the optimal level being about 1.0 wt %. The variables to control the coke on the catalyst include temperature, flow rate of the catalyst, and flow rate of air.

### EXAMPLE 1

In a first experimental example, the catalyst was ZSM-5, which was silylated as described above. For chemical liquid deposition, a ZSM-5 catalyst sample having a mass between 30 and 40 g was suspended in 300-400 ml of n-hexane, and the mixture was heated under stirring until reflux at a temperature between 60° C. and 80° C. After a period of time between 15 and 45 minutes, tetraethyl orthosilicate (TEOS) solution, corresponding to a loading of 2-6 wt % SiO₂, was introduced into the mixture. Silylation was continued for a period of one to three hours at a temperature between 60° C. and 80° C. under reflux and stirring. Excess n-hexane was removed by evacuation. The sample was then dried at a temperature between 80° C. and 100° C. for a period between 20 and 28 hours. The dried sample was then calcined in air at a temperature between 400° C. and 500° C. for between two and six hours at a heating rate of 3-7° C./min. Silylation treatment of the catalyst was repeated between four and eight times using the same procedure. The catalyst was partially coked to enhance the para-diethylbenzene selectivity. The level of coke on the catalyst is preferably in the range of 0.7 wt % to 1.5 wt %, the optimal level being about 1.0 wt %. The amount of coke was maintained by controlled regeneration.

### EXAMPLE 2

In a second example, a fresh sample of the silylated catalyst, as prepared in Example 1, but without partial coking, was tested for disproportionation reaction using ethylbenzene as a feedstock to demonstrate the effectiveness of the catalysts for ethylbenzene disproportionation and production of para-diethylbenzene. A 0.8 g sample of the catalyst was weighed and loaded into a riser simulator basket. The system was then sealed and tested for any pressure leaks by monitoring pressure changes in the system. The catalyst was activated for 15 minutes at 620° C. in a stream of argon. The pre-treatment of the catalyst was carried out using a mixture of ethylbenzene and ethanol (200 μL) in a molar ratio of 1:1 at a reaction temperature of 400° C. for a reaction time of 20 seconds. The system was then purged with argon for ten minutes before the start of the reaction.

Catalytic experiments were carried out in the riser simulator with 200 μL of ethylbenzene injected directly into the reactor via loading syringe for reaction times of 5, 10 and 20 seconds at a 300° C. reaction temperature. The reactor was heated to the desired reaction temperature. The vacuum box was also heated to 250° C. and evacuated to a pressure of 0.5 psi to prevent any condensation of hydrocarbons inside the box. The heating of the riser simulator was conducted under a continuous flow of inert gas (i.e., argon), and it was found that it took a few hours until thermal equilibrium was attained.

The products were analyzed in a gas chromatograph with a flame ionization detector and a capillary column formed from 60-m cross-linked methyl silicone having an internal diameter of 0.32 mm. The product composition is shown below in Table 1, along with para-diethylbenzene selectivity results obtained at a 300° C. reaction temperature without partial coking of the catalyst. The results show about 6-20 wt % conversion of ethylbenzene and a para-diethylbenzene selectivity of 90-95%. The ethylbenzene conversion increases with reaction time, but the para-diethylbenzene selectivity decreases with reaction time.

TABLE 1
Product Composition and para-Diethylbenzene
Selectivity for Example 2
Parameters

| Feedstock | Ethylbenzene | | |
|---|---|---|---|
| Catalyst | Multi-silylated ZSM-5/80 | | |
| Temperature (° C.) | 300 | | |
| Contact Time (s) | 5 | 10 | 20 |
| Catalyst Condition | Fresh | Fresh | Fresh |

Product Composition (wt %)

| | | | |
|---|---|---|---|
| Light Gases | 0.07 | 0.08 | 0.31 |
| Benzene | 3.42 | 5.17 | 9.96 |
| Toluene | 0.06 | 0.11 | 0.32 |
| Ethylbenzene | 93.68 | 90.54 | 81.04 |
| Para-xylene | 0.00 | 0.00 | 0.13 |
| Meta-diethyl benzene | 0.10 | 0.18 | 0.70 |
| Para-diethyl benzene | 2.13 | 3.66 | 6.98 |
| Ortho-diethyl benzene | 0.00 | 0.00 | 0.00 |
| Para-ethyl toluene | 0.00 | 0.06 | 0.19 |
| Ethylbenzene conversion | 6.32 | 9.46 | 18.96 |
| Total diethyl benzenes | 2.23 | 3.84 | 7.67 |
| p-DEB selectivity | 95.43 | 95.20 | 90.91 |

### EXAMPLE 3

In a third example, a fresh sample of the catalyst of Example 1 (also without partial coking, as in Example 2) was tested for alkylation reaction using an ethylbenzene and ethanol feed mixture in a molar ratio of 1:1 to demonstrate the effectiveness of the catalyst for ethylbenzene conversion and production of para-diethylbenzene. The reaction temperatures tested were 300° C., 350° C., and 400° C. All other conditions and procedures were identical to those described above with reference to Example 2.

Table 2 below shows the product composition and para-diethylbenzene selectivity for Example 3. Comparing the results shown in Tables 1 and 2, it can be seen that the para-diethylbenzene yield is almost doubled by using ethanol as an alkylating agent. The selectivity of para-diethylbenzene at 300° C. was greater than 94% in the product obtained after five seconds. However, increasing the contact time to 20 seconds resulted in a decrease in selectivity of less than 93%. Reproducibility runs conducted for 20 seconds at 300° C. showed consistent selectivity within 0.3%. Increasing the reaction temperature to 350° C. and 400° C. resulted in a significant decrease in ethylbenzene conversion, as well as in para-diethylbenzene selectivity. Thus, a temperature of 300° C. is found to be a suitable reaction temperature to achieve higher catalytic activity and selectivity.

TABLE 2
Product Composition and para-Diethylbenzene Selectivity for Example 3
Parameters

| Feedstock | Ethylbenzene:Ethanol = 1:1 molar | | | | | | | | |
|---|---|---|---|---|---|---|---|---|---|
| Catalyst | Multi-silylated ZSM-5/80 | | | | | | | | |
| Temperature (° C.) | 300 | | | | | | 350 | | 400 |
| Contact Time (s) | 5 | 10 | 15 | 20 | 20 | 20 | 20 | 20 | 20 |
| | | | | | (Repeat) | (Repeat) | | (Repeat) | |
| Catalyst Condition | Fresh | Fresh | Fresh | Fresh | Fresh | Fresh | Fresh | Fresh | Fresh |

Product Composition (wt %)

| | | | | | | | | | |
|---|---|---|---|---|---|---|---|---|---|
| Light Gases | 3.02 | 5.56 | 6.73 | 7.26 | 7.72 | 7.49 | 10.77 | 10.17 | 14.44 |
| Ethanol | 18.61 | 13.25 | 10.67 | 8.07 | 8.18 | 8.66 | 7.45 | 7.81 | 8.96 |
| Benzene | 1.13 | 2.67 | 3.84 | 3.76 | 4.57 | 4.22 | 9.82 | 8.60 | 20.81 |
| Toluene | 0.38 | 0.96 | 1.38 | 1.35 | 1.97 | 1.55 | 4.03 | 3.60 | 8.13 |
| Ethylbenzene | 71.32 | 66.06 | 63.41 | 63.16 | 61.60 | 62.03 | 56.12 | 57.63 | 42.47 |
| Para-xylene | 0.13 | 0.29 | 0.40 | 0.37 | 0.51 | 0.42 | 1.08 | 0.99 | 1.53 |
| Meta-xylene | 0.00 | 0.00 | 0.00 | 0.00 | 0.00 | 0.00 | 0.07 | 0.06 | 0.26 |
| Cumene | 0.00 | 0.00 | 0.00 | 0.00 | 0.00 | 0.00 | 0.00 | 0.00 | 0.07 |
| Meta-diethyl benzene | 0.00 | 0.00 | 0.00 | 0.00 | 0.06 | 0.06 | 0.17 | 0.15 | 0.25 |
| Para-diethyl benzene | 0.27 | 0.61 | 0.88 | 0.98 | 1.02 | 1.04 | 1.30 | 1.24 | 0.58 |
| Ortho-diethyl benzene | 4.80 | 9.71 | 11.51 | 13.79 | 12.96 | 13.21 | 7.38 | 7.93 | 1.28 |
| Para-ethyl toluene | 0.00 | 0.00 | 0.00 | 0.00 | 0.00 | 0.00 | 0.00 | 0.00 | 0.05 |
| Meta-ethyl toluene | 0.35 | 0.88 | 1.19 | 1.26 | 1.41 | 1.33 | 1.82 | 1.80 | 0.93 |
| Ethylbenzene Conversion | 15.30 | 21.54 | 24.69 | 24.99 | 26.84 | 26.33 | 33.35 | 31.56 | 49.56 |
| Total diethyl benzenes | 5.07 | 10.32 | 12.38 | 14.77 | 13.98 | 14.25 | 8.68 | 9.18 | 1.91 |
| p-DEB Selectivity | 94.65 | 94.06 | 92.91 | 93.36 | 92.68 | 92.72 | 85.01 | 86.46 | 66.90 |

### EXAMPLE 4

In a fourth example, the catalyst of Example 1 was tested for alkylation reaction using an ethylbenzene and ethanol feed mixture in a molar ratio of 1:1 to demonstrate the effectiveness of the catalyst for ethylbenzene conversion and production of para-diethylbenzene. The reaction temperatures tested were 300° C., 350° C., and 400° C. The catalyst was partially coked using an equimolar mixture of ethylbenzene and ethanol as a feedstock for 20 seconds. All other conditions and procedures were identical to those of Example 2.

As can be seen below in Table 3, the results showed that para-diethylbenzene selectivity was significantly increased when the partially coked catalyst was used. The increase in para-diethylbenzene selectivity was greater than 5% at 300° C., greater than 9% at 350° C., and greater than 15% at 400° C. The yield of total diethylbenzenes was also significantly increased at 350° C. (over 75%) and 400° C. (over 350%) in the product obtained after 20 seconds. However, the absolute value of ethylbenzene conversion was found to be low at 400° C. Reproducibility runs conducted for 20 seconds at 350° C. showed consistent selectivity within 0.5%. Comparing the data in Tables 2 and 3 at reaction temperatures of 300° C. and 350° C. shows that using partially coked multi-silylated ZSM-5/80 catalyst resulted in high para-diethylbenzene selectivity (over 95%) and high para-diethylbenzene yield.

TABLE 3
Product Composition and para-Diethylbenzene Selectivity for Example 4
Parameters

| Feedstock | Ethylbenzene:Ethanol = 1:1 molar | | | | | | | |
|---|---|---|---|---|---|---|---|---|
| Catalyst | Multi-silylated ZSM-5/80 | | | | | | | |
| Temperature (° C.) | 300° C. | | 350° C. | | | | 400° C. | |
| Catalyst Condition | Fresh | Partially | Fresh | Partially | Fresh | Partially | Fresh | Partially |
| | | Coked | | Coked | | Coked | | Coked |
| Contact Time (s) | 20 | 20 | 20 | 20 | 20 | 20 | 20 | 20 |

Product Composition (wt %)

| | | | | | | | | |
|---|---|---|---|---|---|---|---|---|
| Light Gases | 7.49 | 6.32 | 10.77 | 7.16 | 10,17 | 7.46 | 14.44 | 11.23 |
| Ethanol | 8.66 | 6.80 | 7.45 | 7.98 | 7.81 | 8.82 | 8.96 | 7.51 |
| Benzene | 4.22 | 0.72 | 9.82 | 3.08 | 8.60 | 2.82 | 20.81 | 9.06 |
| Toluene | 1.55 | 0.31 | 4.03 | 1.14 | 3.60 | 1.05 | 8.13 | 3.95 |
| Ethylbenzene | 62.03 | 72.32 | 56.12 | 63.05 | 57.63 | 60.36 | 42.47 | 56.07 |
| Para-xylene | 0.42 | 0.16 | 1.08 | 0.37 | 0.99 | 0.36 | 1.53 | 1.10 |
| Meta-xylene | 0.00 | 0.00 | 0.07 | 0.00 | 0.06 | 0.00 | 0.26 | 0.08 |
| Ortho-xylene | 0.00 | 0.00 | 0.00 | 0.00 | 0.00 | 0.00 | 0.07 | 0.00 |
| Cumene | 0.06 | 0.00 | 0.17 | 0.00 | 0.15 | 0.00 | 0.25 | 0.16 |
| Meta-diethyl benzene | 1.04 | 0.25 | 1.30 | 0.84 | 1.24 | 0.84 | 0.58 | 1.09 |
| Para-diethyl benzene | 13.21 | 12.54 | 7.38 | 15.21 | 7.93 | 17.05 | 1.28 | 7.41 |
| Ortho-diethyl benzene | 0.00 | 0.00 | 0.00 | 0.00 | 0.00 | 0.00 | 0.05 | 0.51 |
| Para-ethyl toluene | 1.33 | 0.58 | 1.82 | 1.19 | 1.80 | 1.23 | 0.93 | 1.84 |
| Meta-ethyl toluene | 0.00 | 0.00 | 0.00 | 0.00 | 0.00 | 0.00 | 0.23 | 0.00 |
| Ethylbenzene Conversion | 26.33 | 14.11 | 33.35 | 25.12 | 31.56 | 28.31 | 49.56 | 33.40 |
| Total diethyl benzenes | 14.65 | 13.16 | 8.86 | 16.48 | 9.44 | 16.61 | 1.97 | 9.21 |
| p-DEB Selectivity | 92.72 | 98.04 | 85.01 | 94.78 | 86.46 | 95.32 | 66.90 | 82.30 |

### EXAMPLE 5

The catalyst of Example 1 was tested for alkylation reaction using an ethylbenzene and ethanol feed mixture in a molar ratio of 1:1 to demonstrate the effectiveness of the catalyst for ethylbenzene conversion and production of para-diethylbenzene. The reaction temperature was 300° C. The catalyst was partially coked once or twice using an equimolar mixture of ethylbenzene and ethanol as a feedstock for 20 seconds. All other conditions and procedures were identical to those of Example 2.

As shown below in Table 4, the results showed that para-diethylbenzene selectivity was increased by 4% when the partially coked catalyst was used. Repeating the partial coking of the catalyst twice resulted in almost no change in para-diethylbenzene selectivity. However, the para-diethylbenzene yield dropped due to partial coking and repeated partial coking. Comparing the data in Tables 3 and 4 at a reaction temperature of 300° C., it can be seen that controlled partial coking of multi-silylated ZSM-5/80 catalyst is required for high para-diethylbenzene selectivity and a high para-diethylbenzene yield. The level of coke on the catalyst is preferably in the range of 0.7 wt % to 1.5 wt %, the preferred level being about 1.0 wt %.

TABLE 4
Product Composition and para-Diethylbenzene
Selectivity for Example 5
Parameters

| Feedstock | Ethylbenzene:Ethanol = 1:1 molar | | |
|---|---|---|---|
| Catalyst | Multi-silylated ZSM-5/80 | | |
| Temperature (° C.) | 300 | | |
| Catalyst Condition | Fresh | Partial | Partial |
| | | Coking: | Coking: |
| | | Once | Twice |
| Contact Time (s) | 20 | 20 | 20 |

Product Composition (wt %)

| | | | |
|---|---|---|---|
| Light Gases | 6.77 | 6.80 | 7.96 |
| Ethanol | 8.34 | 8.58 | 7.64 |
| Benzene | 3.16 | 0.57 | 0.28 |
| Toluene | 1.12 | 0.30 | 0.24 |
| Ethylbenzene | 63.91 | 70.92 | 74.64 |
| Para-xylene | 0.38 | 0.16 | 0.19 |
| Meta-diethyl benzene | 0.00 | 0.00 | 0.00 |
| Para-diethyl benzene | 0.00 | 0.00 | 0.00 |
| Ortho-diethyl benzene | 0.00 | 0.00 | 0.00 |
| Para-ethyl toluene | 0.97 | 0.21 | 0.16 |
| Ethylbenzene Conversion | 24.10 | 15.77 | 11.35 |
| Total Diethyl benzenes | 15.66 | 12.58 | 8.78 |
| p-DEB Selectivity | 93.63 | 98.25 | 98.12 |

### EXAMPLE 6

The catalyst of Example 1 was tested for alkylation reaction using an ethylbenzene and ethanol feed mixture in molar ratios of 1:2 and 2:1 to demonstrate the effectiveness of the catalyst for ethylbenzene conversion and production of para-diethylbenzene. A reaction temperature of 300° C. was used. The catalyst was either fresh, partially coked once, or partially coked twice using an equimolar mixture of ethylbenzene and ethanol as a feedstock for 20 seconds. All other conditions and procedures were identical to those of Example 2.

The results, given below in Table 5, show that para-diethylbenzene selectivity was increased by 4% when the partially coked catalyst was used with a 1:2 molar ratio of ethylbenzene and ethanol as the feedstock. However, the ethylbenzene conversion and para-diethylbenzene yield dropped due to partial coking. When the controlled pre-coking of the catalyst was repeated twice and the feedstock was ethylbenzene and ethanol in a 2:1 molar ratio, the ethylbenzene conversion dropped to almost zero. This indicates the importance of controlled partial coking.

TABLE 5
Product Composition and para-Diethylbenzene
Selectivity for Example 6
Parameters

| Feedstock | Ethyl- | | Ethyl- |
|---|---|---|---|
| | benzene:Ethanol = | | benzene:Ethanol = |
| | 1:2 molar | | 2:1 molar |
| Catalyst | Multi-silylated ZSM-5/80 | | |
| Temperature (° C.) | 300 | | |
| Catalyst Condition | Fresh | Partial | Partial |
| | | Coking: | Coking: |
| | | Once | Twice |

Product Composition (wt. %)

| | | | |
|---|---|---|---|
| Gases | 13.06 | 14.28 | 4.51 |
| Ethanol | 12.13 | 14.78 | 3.85 |
| Benzene | 2.19 | 0.32 | 0.26 |
| Toluene | 1.21 | 0.30 | 0.24 |
| Ethylbenzene | 54.95 | 58.32 | 84.15 |
| Para-xylene | 0.57 | 0.26 | 0.30 |
| Meta-diethyl benzene | 0.74 | 0.17 | 0.16 |
| Para-diethyl benzene | 13.73 | 10.75 | 6.16 |
| Ortho-diethyl benzene | 0.00 | 0.00 | 0.00 |
| Para-ethyl toluene | 1.43 | 0.81 | 0.37 |
| EB Conversion | 34.74 | 30.73 | 0.06 |
| Total DEBs | 15.24 | 11.75 | 6.38 |
| p-DEB Selectivity | 94.91 | 98.48 | 97.53 |

It is to be understood that the present invention is not limited to the embodiments described above, but encompasses any and all embodiments within the scope of the following claims.

1. A method of making para-diethylbenzene, comprising steps of:
silylating a zeolite catalyst to form a silylated zeolite catalyst, wherein the step of silylating the zeolite catalyst comprises:
suspending the zeolite catalyst in n-hexane solution to form a mixture;
stirring and heating the mixture until reflux at a temperature between approximately 60° C. and approximately 80° C.;
adding tetraethyl orthosilicate solution to the mixture to silylate the zeolite catalyst for a time period of between approximately one and approximately three hours at a temperature between approximately 60° C. and approximately 80° C. under reflux;
removing excess n-hexane;
drying the silylated zeolite catalyst at a temperature between approximately 80° C. and approximately 100° C. for a time period between approximately 20 and approximately 28 hours; and
calcining the silylated zeolite catalyst in air at a temperature between approximately 400° C. and approximately 500° C. for a time period between approximately two and approximately six hours at a heating rate of approximately 3 to approximately 7° C./min;
partially coking the silylated zeolite catalyst to form a treated catalyst;
contacting a feedstock mixture of ethylbenzene and ethanol with the treated catalyst to produce a reaction product, the feedstock being in a vapor phase;
collecting the reaction product; and
separating para-diethylbenzene from the reaction product.

2. The method of making para-diethylbenzene as recited in claim 1, wherein the zeolite catalyst comprises a zeolite catalyst selected from the group consisting of ZSM-5, ZSM-11, ZSM-12, ZSM-22 and ZSM-23.

3. The method of making para-diethylbenzene as recited in claim 2, wherein the zeolite catalyst has a silica-to-alumina molar ratio between about 50 and about 150,

4. The method of making para-diethylbenzene as recited in claim 1, wherein the zeolite catalyst comprises ZSM-5 zeolite catalyst.

5. The method of making para-diethylbenzene as recited in claim 4, wherein said contacting step comprises contacting the feedstock with the treated catalyst at a reaction temperature between about 250° C. and about 450° C.

6. The method of making para-diethylbenzene as recited in claim 4, wherein said contacting step comprises contacting the feedstock with the treated catalyst at a reaction temperature of about 300° C.

7. The method of making para-diethylbenzene as recited in claim 6, wherein the feedstock mixture of ethylbenzene and ethanol comprises a mixture of ethylbenzene and ethanol in a molar ratio of 1 to 1.

8. The method of making para-diethylbenzene as recited in claim 7, wherein the step of partially coking the silylated zeolite catalyst comprises forming a level of coke on the silylated zeolite catalyst of about 1.0 wt %.

9. The method of making para-diethylbenzene as recited in claim 8, wherein said contacting step further comprises contacting the feedstock with the treated catalyst at a pressure between 1.0 atmospheres and 5.0 atmospheres.

10. The method of making para-diethylbenzene as recited in claim 8, wherein said contacting step further comprises contacting the feedstock with the treated catalyst at a pressure of about 1.0 atmospheres.

11. The method of making para-diethylbenzene as recited in claim 1, wherein the step of silylating the zeolite catalyst comprises multiple silylation of the zeolite catalyst between four and eight times.

12. The method of making para-diethylbenzene as recited in claim 1, wherein said step of separating para-diethylbenzene from the reaction product comprises distilling the reaction product.

13. The method of making para-diethylbenzene as recited in claim 12, further comprising the step of recycling a non-reacted portion of the feedstock mixture.

14. A method of making para-diethylbenzene, comprising steps of:
silylating a zeolite catalyst to form a silylated zeolite catalyst, wherein the step of silylating the zeolite catalyst comprises:
suspending the zeolite catalyst in n-hexane solution to form a mixture;
stirring and heating the mixture until reflux at a temperature between approximately 60° C. and approximately 80° C.;
adding tetraethyl orthosilicate solution to the mixture to silylate the zeolite catalyst for a time period of between approximately one and approximately three hours at a temperature between approximately 60° C. and approximately 80° C. under reflux;
removing excess n-hexane;
drying the silylated zeolite catalyst at a temperature between approximately 80° C. and approximately 100° C. for a time period between approximately 20 and approximately 28 hours; and
calcining the silylated zeolite catalyst in air at a temperature between approximately 400° C. and approximately 500° C. for a time period between approximately two and approximately six hours at a heating rate of approximately 3 to approximately 7° C./min;
partially coking the silylated zeolite catalyst to form a treated catalyst;
contacting a feedstock mixture of ethylbenzene and ethanol in a molar ratio of 1:1 with the treated catalyst at a temperature of about 300° C. to produce a reaction product, the feedstock being in a the vapor phase;
collecting the reaction product; and
separating para-diethylbenzene from the reaction product.

15. The method of making para-diethylbenzene as recited in claim 14, wherein the zeolite catalyst comprises a zeolite catalyst selected from the group consisting of ZSM-5, ZSM-11, ZSM-12, ZSM-22 and ZSM-23.

16. The method of making para-diethylbenzene as recited in claim 14, wherein the zeolite catalyst comprises ZSM-5 zeolite catalyst.

17. The method of making para-diethylbenzene as recited in claim 16, wherein the step of partially coking the silylated zeolite catalyst comprises forming a level of coke on the silylated zeolite catalyst of about 1.0 wt %.

18. The method of making para-diethylbenzene as recited in claim 17, wherein said contacting step further comprises contacting the feedstock with the treated catalyst at a pressure between 1.0 atmospheres and 5.0 atmospheres.

19. The method of making para-diethylbenzene as recited in claim 18, wherein said step of separating para-diethylbenzene from the reaction product comprises distilling the reaction product.
